(19) United States
(12) Patent Application Publication
(10) Pub. No.: US 2016/0206823 A1
(43) Pub. Date: Jul. 21, 2016

(54) ASSEMBLY FOR A DRUG DELIVERY DEVICE AND A METHOD FOR ASSEMBLING A DRUG DELIVERY DEVICE

(71) Applicant: SANOFI-AVENTIS DEUTSCHLAND GMBH, Frankfurt am Main (DE)

(72) Inventors: Michael Jugl, Frankfurt am Main (DE); Alexander Foerster, Frankfurt am Main (DE)

(21) Appl. No.: 14/913,213

(22) PCT Filed: Aug. 15, 2014
(86) PCT No.: PCT/EP2014/067470

§ 371 Date: Feb. 19, 2016

(30) Foreign Application Priority Data
Aug. 22, 2013 (EP) 13181274.5

(51) Int. Cl. A61M 5/315; A61M 5/31; A61M 5/24

(52) U.S. Cl. CPC A61M 5/31511; A61M 5/24; A61M 5/3146; A61M 2005/2403; A61M 2207/00

(57) ABSTRACT

The present invention concerns an assembly for a drug delivery device (1) comprising a drive assembly (3) and a cartridge holder (41). The drive assembly (3) comprises a first engagement member (43) and the cartridge holder (41) comprises a second engagement member (44). The drive assembly (3) is configured to be arranged inside a body (2) of the drug delivery device (1). The drive assembly (3) is engageable to the cartridge holder (41) by an engagement of the first and the second engagement member (43, 44). Moreover, the present invention concerns a method for assembling the drug delivery device (1).

The present invention relates to an assembly for a drug delivery device. Further, the present invention concerns a method for assembling a drug delivery device.

Drug delivery devices are generally known for the administration of a medicinal product, for example insulin or heparin, but also for other medicinal products, in particular for self-administration by a patient. A drug delivery device may be configured as a pen-type injector, which may dispense a pre-set dose of a fluid medicinal product. However, the drug delivery device may also deliver a variable dose of a medicinal product.

Before the first use of the drug delivery device, the user may have to dispense a small amount of the product. Thereby, the drug delivery device may be adjusted to manufacturing tolerances of its mechanical components. The operation of dispensing a small amount of the product before the first use is also referred to as a priming operation of the drug delivery device. Users who are unfamiliar with the drug delivery device may fail or incorrectly prime their drug delivery device before dispensing the first dose. Another disadvantage of a drug delivery device requiring a priming operation is that a user might accidentally inject a priming dose. Moreover, the priming operation results in a waste of a medicinal product as the medicinal product expelled during the priming operation can not be used to treat the patient.

WO 2011/039215 A1 discloses a drug delivery device which does not require a priming step.

It is an object of the present disclosure to provide an assembly for use in a drug delivery device which helps to improve usability and ensures the accuracy of the first administered dose of a medicinal product.

This object is solved by the assembly according to present claim 1. Further, the object is also solved by the method for assembling the drug delivery device according to the further independent claim.

According to a first aspect of the present disclosure, an assembly for a drug delivery device is provided which comprises a drive assembly and a cartridge holder. The drive assembly comprises a first engagement member and the cartridge holder comprises a second engagement member. The drive assembly may be configured to be arranged inside a body of the drug delivery device. The drive assembly is engageable to the cartridge holder by an engagement of the first and the second engagement member.

In particular, the assembly may comprise elements corresponding structurally and functionally to the components of the dosing and drive mechanism disclosed by WO 2008/058665 A1. In particular, the drive assembly may comprise elements corresponding structurally and functionally to the piston rod and the drive sleeve of the dosing and drive mechanism disclosed by WO 2008/058665 A1. Further, the cartridge holder may correspond structurally and functionally to the cartridge holder disclosed by WO 2008/058665 A1.

The drive assembly may be configured for transferring force, preferably user-exerted force, particularly preferably manually exerted force, to other elements of the drug delivery device for expelling the medicinal product. In particular, the drive assembly may be configured for transferring the force to a bung of a cartridge which is arranged in the cartridge holder, thereby displacing the bung with respect to the cartridge in a dose dispensing operation in a distal direction.

The terms “distal” and “proximal” shall be defined as follows. In an assembled drug delivery device, the distal end of the drive assembly is defined as the end which is closest to a dispensing end of the drug delivery device. In an assembled drug delivery device, the proximal end of the drive assembly is defined as the end which is furthest away from the dispensing end of the drug delivery device. Moreover, a distal direction is defined as a direction towards the distal end and a proximal direction is defined as a direction towards the proximal end.

The drive assembly may be engaged to the cartridge holder before being inserted into the body. Accordingly, the first and the second engagement members may be configured to engage the drive assembly and the cartridge holder to each other without mechanical interaction with the body and without mechanical support from the body.

When the drive assembly and the cartridge holder are engaged to each other, a priming elimination step may be carried out before inserting the assembly into the body. In particular, in this step, an element of the drive assembly may be aligned to an element of the cartridge holder. Thereby, manufacturing tolerances and other mechanical tolerances may be adjusted. In particular, in the priming elimination step, a piston rod of the drive assembly may be aligned to the bung of the cartridge arranged inside the cartridge holder either by bringing the piston rod into abutment with the bung or by arranging the piston rod at a pre-defined distance to the bung.

The engagement of the first and the second engagement members may allow carrying out the priming elimination step before inserting the assembly into the body. Accordingly, it may be possible to control this step by optical monitoring. In particular, optical monitoring of this step is not prevented by the body. If the priming elimination step is optically monitored, its accuracy is increased.

Moreover, the drive assembly being engaged to the cartridge holder by an engagement of the first and the second engagement members may allow the construction of a drug delivery device wherein manufacturing tolerances of the body do not interfere with the operation of the drug delivery device, i.e. with a dose setting operation and/or a dose delivery operation. In particular, the manufacturing tolerances of the body may not have to be equaled out in the priming elimination step as they may not influence the later operation of the drug delivery device. Therefore, the tolerance chain of the drug delivery device may be reduced, thereby making the device more reliable and less susceptible to manufacturing errors.

Further, the drive assembly being engaged to the cartridge holder by an engagement of the first and the second engagement members may allow the construction of a drug delivery device wherein a force applied by a user to a button of the device is transferred more directly to the cartridge holder, in particular more directly to the bung of the cartridge arranged in the cartridge holder. In particular, the force may be transferred without interaction with the body in which the drive assembly is arranged. The direct transfer of the force results in an increase in the dosing accuracy. Moreover, it may result in a reduction of the force that a user has to apply in order to initiate a dose delivery operation. This is particularly important for older users and for users with limited manual skills.

The drive assembly may comprise a piston rod comprising a first piston rod member and a second piston rod member. In a first state of the drive assembly, the first and the second piston rod member are moveable with respect to each other to adjust the length of the piston rod.

The first state of the drive assembly may be a pre-assembled state of the drug delivery device. In the first state, the drive assembly may be engaged to the cartridge holder holding a cartridge. Moreover, the drive assembly may not have been arranged inside the body of the drug delivery device in the first state.

In the first state, manufacturing tolerances or other mechanical tolerances may exist such that the drive assembly may be not correctly aligned to other elements of the drug delivery device. In particular, the distance between elements of the drive assembly, for example the piston rod, and the bung of the cartridge arranged in the cartridge holder may not be well defined. Due to mechanical tolerances and other tolerances in the manufacturing process this distance may vary from one drug delivery device to another in the first state of the drive assembly.

Due to the mechanical tolerances in the first state, the drive assembly may be usable for preparative purposes only and may be not suited for a dose setting operation or a dose dispensing operation before the tolerances have been adjusted.

The first piston rod member may be configured as a threaded insert. In particular, the first piston rod member may be configured as a threaded insert which is inserted into the second piston rod member. The second piston rod member may comprise a lead screw.

The drive assembly may further have a second state, in which the first piston rod member is fixed to the second piston rod member to define a fixed length piston rod. Further, in the second state, the drive assembly may be arranged inside the body.

Moreover, the drug delivery device may have a partially-assembled and a fully-assembled state. In the partially-assembled state of the drug delivery device, the drive assembly may be not arranged in the body. In the partially-assembled state of the drug delivery device, the drive assembly may be in its first or its second state. In the fully-assembled state of the drug delivery device, the drive assembly may be arranged in the body. In the fully-assembled state of the drug delivery device, the drive assembly may be in its second state.

In its second state, the drive assembly may be used by a patient for the application of a medicament. Accordingly, the drive assembly may be ready to carry out a dose setting and a dose dispensing operation.

In particular, when the drive assembly is operated for the first time in the second state, the drive assembly can be used without the requirement of a priming step to prepare for the first dose delivery.

Further, the drive assembly may comprise a fixed member which is configured to be not moveable relative to the cartridge holder when the first and the second engagement members are engaged.

The fixed member may be a nut. Moreover, the fixed member may comprise the first engagement member. Further, the fixed member may be configured not to be moveable relative to the body when the drive assembly is arranged inside the body.

Further, the drive assembly may comprise a moveable member which is configured to be moveable relative to the cartridge holder when the first and the second engagement members are engaged.

The moveable member may comprise at least one of the piston rod, an adjustment member and a drive member. The drive member may be a drive sleeve. The adjustment member may be configured to move the first piston rod member relative to the second piston rod member in the first state of the drive assembly. Moreover, the moveable member may be configured to be moveable relative to the body when the drive assembly is arranged inside the body.

Moreover, the moveable member may be threadedly connected to the fixed member.

In particular, the movable member may comprise the piston rod which is threadedly connected to the fixed member. In particular, the moveable member may be screwed through the fixed member in a dose dispensing operation of the drug delivery device.

Further, the drive assembly comprises a member being at least partially transparent and comprising the first engagement member.

In particular, the at least partially transparent member may be the fixed member. The member being at least partially transparent may allow for an optical monitoring of the priming elimination step. In particular, the member being partially transparent may allow for optically monitoring the distance between the moveable member and the cartridge holder, or more particular, between the moveable member and a bung of a cartridge arranged inside the cartridge holder.

The member may be partially transparent for laser light. Therefore, a laser may pass through the at least partially transparent member. Alternatively or additionally, the member may be at least partially transparent for light with a wavelength in the range of visible light.

Moreover, the cartridge holder may be at least partially transparent. This may also allow for an optical monitoring of the priming elimination step. In particular, the cartridge holder may be partially transparent for laser light and/or for light with a wavelength in the range of visible light.

The first and the second engagement members may be configured to be positively locked to each other. A positive locking may be constructed such that the engagement members are unlikely to work loose from vibrations which may occur during manufacturing or operation of the drug delivery device. The positive lock between the first and the second engagement members may require only small forces to engage the first and the second engagement members to each other.

The first and the second engagement member may be configured to form a clip engagement. In particular, one of the first or the second engagement member may comprise an opening. Further, the other of the first and the second engagement member may comprise a protrusion adapted to engage into the opening of the one of the first or the second engagement member.

In particular, the protrusion may be shaped, e.g. tapered, such that it may be inserted into the opening with a force which is significantly lower than the force required to remove the protrusion from the opening.

Alternatively or additionally, the first and the second engagement member may be configured to form a bayonet engagement. Accordingly, only a small force may be required to engage the first and the second engagement member to each other.

Another aspect of the present disclosure relates to a drug delivery device which comprises the assembly and a body, wherein the body accommodates the drive assembly.

In particular, the assembly may be the assembly disclosed above such that every structural and functional feature disclosed with respect to that assembly may also be present in the drug delivery device.

The body may comprise an opening such that the drive assembly may be arranged inside the opening.

Moreover, the body may be configured such that the cartridge holder is not connected to the body. Instead, the cartridge holder may be connected only to the drive assembly. Moreover, the body may be configured such that, any tolerances in the manufacturing of the body may not affect the operation of the drive assembly. Accordingly, the accuracy of the drive assembly may be increased, thereby improving the dosing accuracy of the drug delivery device.

For example, the body may be connected to an assembly comprising the drive assembly and the cartridge holder by clipping. Alternatively or additionally, the body may be connected to the assembly comprising the drive assembly and the cartridge holder by welding or by adhesive bonding.

Another aspect of the present invention relates to a method for assembling a drug delivery device. The method comprises the steps of:
providing a drive assembly and a cartridge holder, wherein the drive assembly is configured to be arranged inside a body, and wherein the drive assembly comprises a first engagement member and the cartridge holder comprises a second engagement member, and
engaging the drive assembly to the cartridge holder by an engagement of the first and the second engagement member.

In particular, the method may be used to assemble a drug delivery device comprising the assembly as disclosed above. Accordingly, every structural and functional feature disclosed with respect to that assembly or the drug delivery device may also be present in the method.

The drive assembly may comprise a piston rod comprising a first piston rod member and a second piston rod member, wherein in a first state of the drive assembly, the first and the second piston rod member are moveable with respect to each other to adjust the length of a piston rod, and wherein the method further comprises the step of adjusting the length of the piston rod after engaging the drive assembly to the cartridge holder.

Further, a cartridge comprising a bung may be arranged inside the cartridge holder. The step of adjusting the length of the piston rod may comprise the sub-step of fixing the first piston rod member to the second piston rod member when the distance between the piston rod and the bung equals a predefined value or when the piston rod abuts the bung, thereby forming a fixed length piston rod.

The step of adjusting the length of the piston rod may comprise the sub-step of visually controlling the distance between the first piston rod member and the bung. The distance may be monitored using a laser. This allows for a very accurate measurement of the distance.

The method may further comprise the steps of providing a body, and of arranging the drive assembly in the body after the length of the piston rod has been adjusted.

The term “medicinal product”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly- Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 Exendin-4(1-39),

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

In the following, the disclosed devices and methods are described in further detail with reference to the drawings, wherein

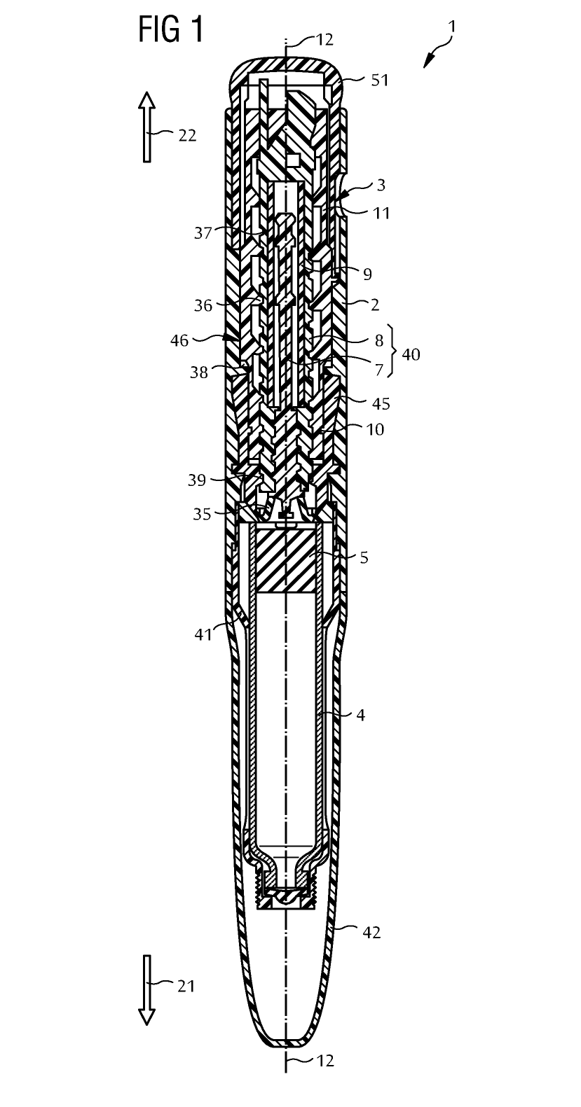

FIG. 1 shows a drug delivery device,

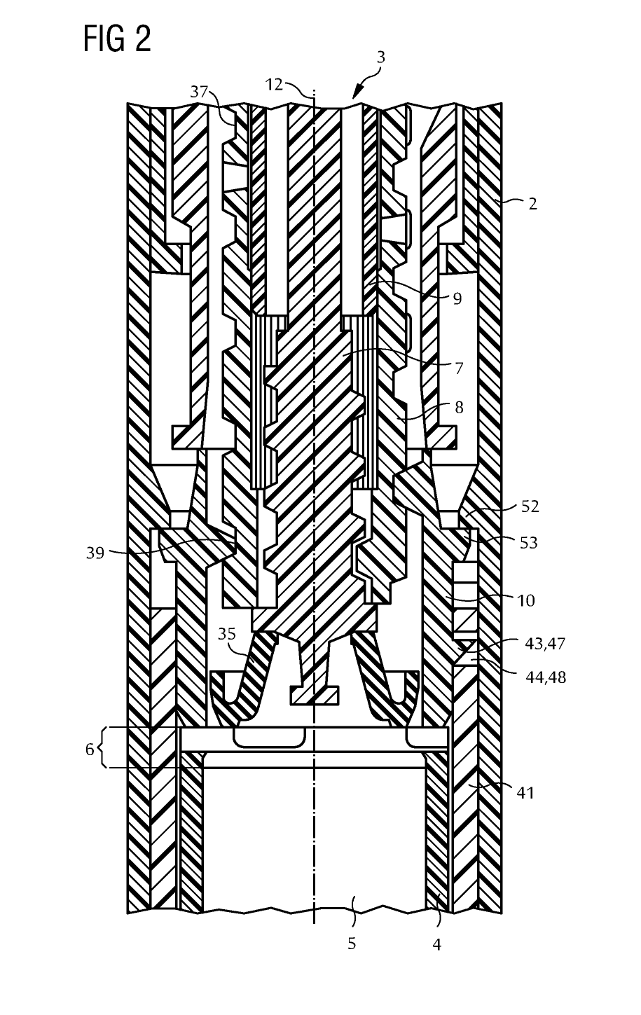

FIG. 2 shows an enlarged view of a part of the drug delivery device shown in FIG. 1.

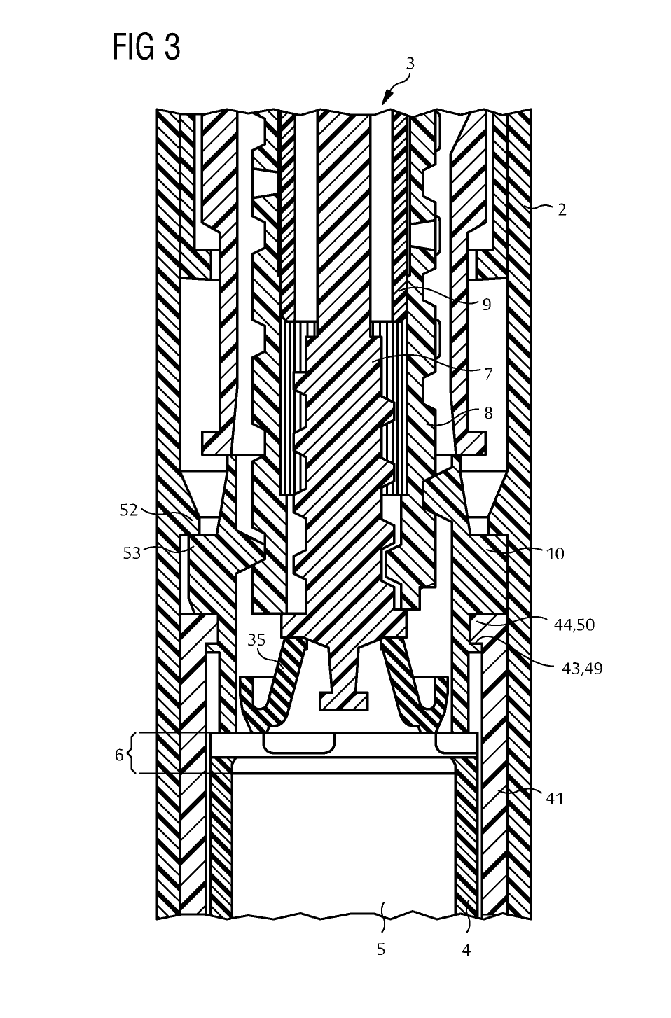

FIG. 3 shows an enlarged view of a part of the drug delivery device according to an alternate embodiment.

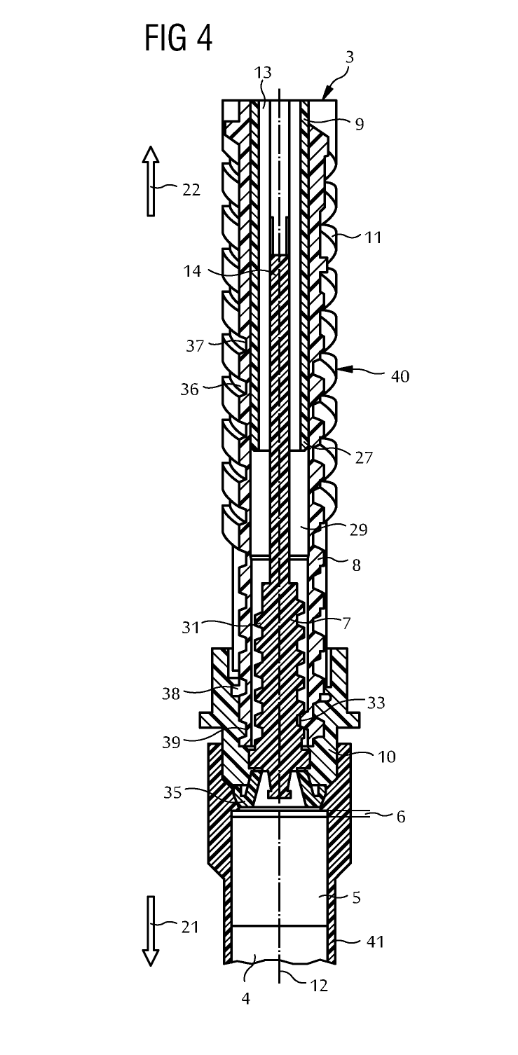

FIG. 4 shows a part of the drug delivery device shown in FIG. 1 in a first state.

FIG. 1 shows a cross-sectional view of a drug delivery device 1. In particular, FIG. 1 shows the drug delivery device 1 after the assembly of the drug delivery device 1 has been completed.

The drug delivery device 1 shown in FIG. 1 is an injection device. In particular, the drug delivery device 1 is a pen-type injection device. Moreover, the drug delivery device 1 is a fixed dose device. The drug delivery device 1 is a disposable device.

The drug delivery device 1 comprises a body 2, a drive assembly 3 and a cartridge holder 41. The drive assembly 3 is arranged at least partially inside the body 2. Moreover, the cartridge holder 41 may also be arranged at least partially inside the body 2. In the embodiment shown in FIG. 1, the proximal end part of the cartridge holder 41 is arranged in the body 2. Further, a cartridge 4 comprising a bung 5 and a medicinal product is arranged inside the cartridge holder 41. By a movement of the bung 5 in a direction towards an outlet of the cartridge 4 a medicament may be expelled from the cartridge 4.

Moreover, the drug delivery device 1 comprises a cap 42. The cap 42 is configured to cover the part of the cartridge holder 41 which is not arranged inside the body 2. The cap 42 is engageable to the cartridge holder 41, e.g. by a clipping connection. The cap 42 can be removed from the cartridge holder 41. In particular, the cap 42 has to be removed before a needle (not shown) may be attached to the distal end of the cartridge holder 41.

The body 2 is connected to the drive assembly 3 and the cartridge holder 41 such that the cartridge holder 41 is not mechanically connected to the body 2. In other words, the body 2 is directly mechanically connected only to the drive assembly 3. The cartridge holder 41 is prevented from a direct mechanical connection to the body 2. Instead, the cartridge holder 41 is mechanically connected only to the drive assembly 3. Moreover, the body 2 is connected to the drive assembly 3 such that any tolerances in the manufacturing of the body 2 do not affect the drive assembly 3. Accordingly, the accuracy of the drive assembly 3 is increased. Therefore, the dosing accuracy of the drug delivery device 1 is improved.

For example, the body 2 may be connected to an assembly comprising the drive assembly 3 and the cartridge holder 41 by clipping. Alternatively, the body 2 may be connected to the assembly comprising the drive assembly 3 and the cartridge holder 41 by welding or by adhesive bonding.

The drive assembly 3 comprises a fixed member 45 and moveable members 46. The fixed member 45 is configured to be mechanically engaged to the cartridge holder 41. Due to this engagement, the cartridge holder 41 is engaged to the drive assembly 3. However, as the cartridge holder 41 is engaged to the drive assembly 3, the cartridge holder 41 does not need to be engaged to the body 2.

The fixed member 45 is configured to be not moveable relative to the cartridge holder 41 when the drive assembly 3 is engaged to the cartridge holder 41. The fixed member 45 comprises a nut 10.

Moreover, the moveable members 46 are configured to be moveable relative to the cartridge holder 41 when the drive assembly 3 is engaged to the cartridge holder 41. In particular, a piston rod 40 is one of the moveable members 46. The piston rod 40 comprises a first piston rod member 7 and a second piston rod member 8. Each of the first piston rod member 7 and the second piston rod member 8 is moveable relative to the cartridge holder 41. Further, other moveable members 46 of the drive assembly 3 are an adjustment member 9 and a drive member 11.

Moreover, the drug delivery device 1 comprises a button 51 attached to the drive member 11. The button may close the body 2. For dose setting, the button 51 may be pulled in the proximal direction 22 and for dose dispensing the button 51 may be pushed in the distal direction 21.

The first and the second piston rod member 7, 8 are configured to be fixed to each other in the second state of the drive assembly 3. When the first and the second piston rod member 7, 8 are fixed to each other, they form a fixed-length piston rod 40.

The adjustment member 9, the first piston rod member 7 and the second piston rod member 8 are arranged coaxially to a longitudinal axis 12 of the drive assembly 3.

The adjustment member 9 is arranged at least partially inside the second piston rod member 8. In particular, the second piston rod member 8 comprises an opening which extends through the second piston rod member 8 in a longitudinal direction. The opening has a circular cross-section. The adjustment member 9 has a diameter which is smaller than the diameter of the opening defined in the second piston rod member 8. In particular, the adjustment member 9 is arranged at least partially inside the opening extending through the second piston rod member 8.

Further, the adjustment member 9 also comprises an opening which extends through the adjustment member 9 in a longitudinal direction. The first piston rod member 7 is arranged at least partially inside the adjustment member 9. In particular, the first piston rod member 7 is arranged at least partially inside the opening defined by the adjustment member 9. Thereby, the first piston rod member 7 is also arranged at least partially inside the second piston rod member 8.

Further, the first piston rod member 7 comprises a bearing 35 arranged at its distal end. The bearing 35 is configured to abut the bung 5 in the cartridge 4. Furthermore, the bearing 35 of the first piston rod member 7 is adapted to provide a force on the bung 5 such that the bung 5 is moved in a distal direction 21 and a medicinal product is expelled from the cartridge 4.

Moreover, the adjustment member 9, the first piston rod member 7 and the second piston rod member 8 are arranged at least partially inside the drive member 11. The drive member 11 is adapted to be moved in an axial direction. In particular, the drive member 11 can be moved in the distal direction 21 and in the proximal direction 22.

However, the drive member 11 is coupled to the body 2 such that a rotational movement of the drive member 11 relative to the body 2 is prevented.

Further, the second piston rod member 8 comprises a first thread 39 at its distal end and a second thread 37 at its proximal end. The first and the second thread 39, 37 are arranged at the outer surface of the second piston rod member 8.

The second thread 37 comprises a series of part threads, rather than a complete thread. The part threads are formed on flexible arms of the second piston rod member 8.

The first thread 39 and the second thread 37 of the second piston rod member 8 are oppositely disposed.

The nut 10 is arranged at the distal end of the drive member 11. In particular, the nut 10 is provided with a threaded circular opening. The threaded circular opening comprises a series of part threads rather than a complete thread. Additionally, the nut 10 comprises non-return features (not shown), e.g. flat sections in the part threads.

Further, the second piston rod member 8 extends through the nut 10. The second piston rod member 8 is threadedly engaged with the nut 10 by an engagement of the first thread 39 of the second piston rod member 8 and the threaded circular opening of the nut 10.

Moreover, the nut 10 is rotationally and translationally fixed to the body 2. A protrusion of the nut 10 is accommodated in an indention of the body 2 by a clip engagement. In particular, in the inner surface of the body 2, the indentation is formed which is configured to engage with the nut 10. Further, the indentation is formed such that a movement of the nut 10 relative to the body 2 is prevented.

The drive member 11 comprises a thread 36 at its inner surface. The drive member 11 and the second piston rod member 8 are configured such that the thread 36 of the drive member 11 and the second thread 37 of the second piston rod member 8 engage to each other when the drive member 11 is moved in the distal direction 21. Due to the threaded engagement of the drive member 11 and the second piston rod member 8, the second piston rod member 8 carries out a rotational movement, when the drive member 11 is moved in the distal direction 21.

Further, the drive member 11 is configured such that the drive member 11 and the second piston rod member 8 disengage from each other, when the drive member 11 moves in the proximal direction 21. Accordingly, the second piston rod member 8 does not move, when the drive member 11 moves in the proximal direction 21.

In particular, the first thread 39 of the second piston rod member 8 is provided with a plurality of features (not shown) that cooperate with the non-return features of the threaded circular opening of the nut 10 to prevent a movement of the second piston rod member 8 in the proximal direction 21 when the drive member 11 moves in the proximal direction 21.

Moreover, the drive assembly 3 comprises a spring member 38. The spring member 38 is arranged at the proximal end of the nut 10.

The spring member 38 may be tensed by the drive member 11 moving in a distal direction 21 and thereby applying a force on the spring member 38. Once the force applied by the drive member 11 is released, the spring member 38 is allowed to relax. Thereby, the spring member 38 exerts a force on the drive member 11 in a proximal direction, resulting in a small movement of the drive member 11 in the proximal direction 22. This movement also causes a small proximal movement of the second piston rod member 8.

FIG. 2 shows an enlarged view of a part of the drug delivery device as shown in FIG. 1.

The drive assembly 3 comprises a first engagement member 43. In particular, the nut 10 comprises the first engagement member 43. The cartridge holder 41 comprises a second engagement member 44. In FIG. 2, the first and the second engagement member 43, 44 are engaged to each other. Accordingly, the drive assembly 3 is engaged to the cartridge holder 41.

In the embodiment shown in FIG. 2, the first engagement member 43 comprises a protrusion 47. The protrusion 47 is arranged at an outer surface of the nut 10. The protrusion 47 faces away from a longitudinal axis 12 of the drive assembly 3.

Further, the second engagement member 44 of the cartridge holder 41 comprises an opening 48. Further, the protrusion 47 is adapted to engage into the opening 48 defined in the cartridge holder 41. When the protrusion 47 is engaged in the opening 48, the nut 10 is fixed to the cartridge holder 41 such that it cannot move relative to the cartridge holder 41.

The distal face of the protrusion 47 is tapered and the proximal face of the protrusion 47 is roughly perpendicular to the longitudinal axis 12 of the drive assembly 3. Therefore, it is possible to insert the protrusion 47 into the opening 48 easily. Therefore, the protrusion 47 and the opening 48 form a clip engagement.

Further, in the embodiment shown in FIG. 2, the body 2 is engaged to the drive assembly 3 by a clip engagement. In particular, the body 2 comprises an inward facing protrusion 52 and the drive assembly 3 comprises an outward facing protrusion 53. The outward facing protrusion 53 is arranged on the nut 10 of the drive assembly 3. The inward facing protrusion 52 abuts the outward facing protrusion 53, thereby preventing a translational movement of the body 2 relative to the drive assembly 3 in the distal direction 21. Other movements of the body 2 relative to the drive assembly 3, e.g. a rotational movement or a translational movement in the proximal direction, may be prevented by other clipfeatures or by a welding or bonding of the body 2 to the drive assembly 3.

FIG. 3 shows an enlarged view of a part of the drug delivery device 1 according to an alternative embodiment.

In the alternative embodiment, the first engagement member 43 comprises a recess 49. Further, the second engagement member 44 comprises a projection 50 facing from an inner sidewall of the cartridge holder 41 towards the longitudinal axis 12 of the drive assembly 3. The recess 49 of the first engagement member 43 in the nut 10 is shaped such that it forms a bayonet connection with the projection 50 of the cartridge holder 41.

Accordingly, in order to engage the cartridge holder 41 with the drive assembly 3, the cartridge holder 41 needs to be moved firstly in a proximal direction 22 and afterwards rotated. When the projection 50 is engaged to the recess 49, the nut 10 is locked against a translational movement relative to the cartridge holder 41.

Further, in the embodiment shown in FIG. 3, the body 2 is also engaged to the drive assembly 3 by a clip engagement of the inward facing protrusion 52 of the body 2 and the outward facing protrusion 53 of the drive assembly 3.

FIG. 4 shows a part of the drive assembly 3 in a first state.

Moreover, the drug delivery device 1 is in its partially-assembled state. The first state of the drive assembly 3 corresponds to a state in the assembly process wherein, due to manufacturing tolerances, the drive assembly 3 and the cartridge holder 41 have not yet been aligned to each other such that a distance 6 between the drive assembly 3 and the bung 5 is not well-defined. In particular, the distance 6 may vary from one drug delivery device 1 to another at this stage of the assembly process.

In the first state of the drive assembly 3 and in the partially-assembled state of the drug delivery device 1, the following assembling steps have already been carried out:

The first piston rod member 7 has been inserted into the second piston rod member 8, e.g. by screwing the first piston rod member into the second piston rod member 8. In particular, the first piston rod member 7 comprises a connection member 31, e.g. a helical thread at its outer surface. The second piston rod member 8 comprises a connection member 33, e.g. a helical thread at its inner surface. The first and the second piston rod member 7, 8 are connected to each other by connecting the connection member 31 of the first piston rod member 7 and the connection member 33 of the second piston rod member 8, thereby permitting a concurrent relative rotational and translational movement of the first and the second piston rod member 7, 8 and preventing an exclusive rotational movement and an exclusive translational movement of the first and the second piston rod member 7, 8 relative to each other.

Further, the first and the second piston rod members 7, 8 are inserted into the drive member 11. The nut 10 is attached to the distal end of the drive member 11, e.g. by a threaded engagement. The bearing 35 is attached to the distal end of the first piston rod member 7, e.g. by clipping.

The adjustment member 9 is inserted into the second piston rod member 8. Thereby, the adjustment member is engaged to the first piston rod member 7. In particular, the adjustment member 9 comprises a first connection member 13 which engages a further connection member 14 of the first piston rod member 7. The first connection member 13 of the adjustment member 9 comprises the opening defined in the adjustment member 9 wherein this opening has a non-circular, e.g. cross-shaped, shape when viewed in a cross-sectional view in a plane perpendicular to the longitudinal axis 12 of the drive assembly 3. Further, the further connection member 14 of the first piston rod member 7 comprises a part of the first piston rod member 7 having a non-circular cross-section corresponding to the non-circular opening of the first connection member 13. A relative rotational movement of the adjustment member 9 and the first piston rod member 7 is prevented by the non-circular cross-section part of the first piston rod member 7 being arranged inside the non-circular opening defined in the adjustment member 9. Further, the first connection member 13 of the adjustment member 9 and the further connection member 14 of the first piston rod member 7 are configured such that a translational movement of the adjustment member 9 relative to the first piston rod member 7 in a distal direction 21 is permitted when the first and the further connection member 13, 14 are engaged.

Further, the adjustment member 9 comprises a second connection member 27, e.g. comprising splined elements. The second piston rod member 8 comprises a further connection member 29 adapted to be connected with the second connection member 27 of the adjustment member 9. The further connection member 29 of the second piston rod member 8 comprises splined elements arranged in a distal end part of the second piston rod member 8.

In the first state of the drive assembly 3, the second connection member 27 of the adjustment member 9 and the further connection member 29 of the second piston rod member 8 are not connected.

The drive assembly 3 and the cartridge holder 41 may be assembled to each other in the first state by an engagement of the first and the second engaging members 43, 44. However, in the first state, the drive assembly 3 and the cartridge holder 41 may not yet have been arranged inside the body 2.

In the first state, the first connection member 13 of the adjustment member 9 is connected to the further connection member 14 of the first piston rod member 7. Accordingly, in the first state, the first piston rod member 7 has to follow a rotational movement of the adjustment member 9.

Further, in the first state of the drive assembly 3, the second connection member 27 of the adjustment member 9 is not engaged to the further connection member 29 of the second piston rod member 8. Instead, the second connection member 27 and the further connection member 29 are arranged at a distance from each other. Accordingly, in the first state, the second and the further connection members 27, 29 do not prevent any relative movement of the adjustment member 9 and the second piston rod member 8.

One of the operations carried out during an assembly process of the drug delivery device 1 is an adjustment of the length of the piston rod 40. This operation allows adjusting for manufacturing tolerances and other mechanical tolerances. Accordingly, this operation is a priming elimination operation.

A first step of the operation of adjusting the length of the piston rod 40 is moving the drive member 11 in a distal direction. In particular, the drive member 11 is held in the distal position during the whole operation of adjusting the length of the piston rod 40.

When the drive member 11 moves in the distal direction, it abuts the nut 10 and exerts a force on the spring member 38 arranged at the proximal end of the nut 10, thereby the spring member 38 is tensed.

However, this first step of the operation of adjusting the length of the piston rod 40 is optional. It does not have to be carried out to adjust the length of the piston rod 40.

In a further step of the operation of adjusting the length of the piston rod 40, the adjustment member 9 is rotated about the longitudinal axis 12.

Due to the connection of the first connection member 13 and the further connection member 14 of the first piston rod member 7, the first piston rod member 7 is rotationally fixed to the adjustment member 9 such that the first piston rod member 7 follows the rotational movement of the adjustment member 9.

Further, in the first state, the second connection member 27 and the further connection member 29 of the second piston rod member 8 are not connected to each other. Accordingly, a relative rotational and axial movement of the adjustment member 9 relative to the second piston rod member 8 is permitted. In particular, a movement of the adjustment member 9 is not transferred into a movement of the second piston rod member 8.

Accordingly, the first piston rod member 7 following the rotational movement of the adjustment member 9 is rotated relative to the second piston rod member 8.

Furthermore, the connection members 31, 33 of the first and the second piston rod member 7, 8 are connected to each other, thereby allowing only a concurrent and axial movement of the first piston rod member 7 relative to the second piston rod member 8. Accordingly, the rotational movement of the first piston rod member 7 relative to the second piston rod member 8 also triggers a movement of the first piston rod member 7 in a distal direction relative to the second piston rod member 8.

Due to the movement of the first piston rod member 7 in the distal direction, the distance between the second connection member 27 of the adjustment member 9 and the further connection member 29 of the second piston rod member 8 is reduced.

Due to the movement of the first piston rod member 7 in the distal direction, the bearing 35 of the first piston rod member 7 is moved closer to the bung 5 of the cartridge 4.

The distance 6 between the bearing 35 and the bung 5 is optically monitored before the assembly is inserted into the body 2. This optical monitoring may be carried out using laser sensors.

In particular, the nut 10 and the cartridge holder 41 are at least partially transparent to allow for an optical monitoring of the distance 6 between the bearing 35 and the bung 5.

The bearing 35 may be moved in the distal direction 21 until the bearing 35 of the first piston rod member 7 abuts the bung 5. Alternatively, the bearing 35 may be moved in the distal direction 21 until the bearing 35 has a predefined distance to the bung 5.

Additionally or alternatively to the optical monitoring of the distance 6 between the bearing 35 and the bung 5, a detection member (not shown) may be used to detect the abutment of the bearing 35 on the bung 5 and to stop the movement of the first piston rod member 7 in the distal direction 21 in case of abutment. In particular, the detection member may detect an increase in the torque required to rotate the adjustment member 9. This increase is due to the abutment of the bearing 35 and the bung 5.

The detection member may comprise a torque sensor or a slip clutch for this purpose. For example, once the bearing 35 contacts the bung 5, the slip clutch may slip due to the increased torque and, thereby, the rotation of the adjustment member 9 is stopped. This also stops the movement of the first piston rod member 7.

A last step of the adjustment of the length of the piston rod 40 is carried out when an abutment of the bearing 35 and the bung 5 has been detected and when the rotation of the adjustment member 9 has been stopped.

In the last step of the adjustment of the length of the piston rod 40, the adjustment member 9 is pushed inward into the second piston rod member 8. Accordingly, the adjustment member 9 is moved in the distal direction 21 relative to the first piston rod member 7 and relative to the second piston rod member 8.

During this translational movement, the second connection member 27 of the adjustment member 9 and the further connection member 29 of the second piston rod member 8 are connected to each other. Due to this engagement, a rotational movement of the adjustment member 9 relative to the second piston rod member 8 is prevented.

Accordingly, the adjustment member 9 is prevented from a rotational movement relative to the first piston rod member 7 due to the connection of the first connection member 13 of the adjustment member 9 and the further connection member 14 of the first piston rod member 7 and, further, due to the connection of the second connection member 27 and the further connection member 29 of the second piston rod member 8, the adjustment member 9 is prevented from a rotational movement relative to the second piston rod member 8. Accordingly, as the first connection member 13 of the adjustment member 9 and the further connection member 14 of the first piston rod member 7 are connected to each other and the second connection member 27 and the further connection member 29 of the second piston rod member 8 are connected to each other, a relative rotational movement of the first and the second piston rod member 7, 8 is also prevented.

Moreover, due to the engagement of the connection members 31, 33, the first and the second piston rod member 7, 8 are only allowed to carry out concurrent rotational and translational movements relative to each other. As rotational movements are no longer possible, the first and the second piston rod member 7, 8 are thereby also translationally fixed to each other after the last step has been carried out.

Therefore, the first and the second piston rod member 7, 8 define the piston rod 40 of a fixed length. In particular, after the last step of the adjustment of the length of the piston rod 40 has been completed, the length of the piston rod 40 cannot be altered such that the drug delivery device 1 is operated with the piston rod 40 of a fixed length.

In case the optional first step of moving the drive member 11 in the distal direction 21 and of holding the drive member 11 in its distal position is carried out, the last step of the adjustment of the length of the piston rod 40 comprises the sub-step of releasing the drive member 11. Accordingly, the drive member 11 is not held in its distal position anymore. Then, the drive member 11 does not apply a force on the spring member 38 anymore and the spring member 38 is allowed to relax.

When the spring member 38 relaxes, it moves the drive member 11 in the proximal direction 22. Moreover, when the spring member 38 relaxes, this also results in a movement of the second piston rod member 8 in the proximal direction 22. Thereby, the complete fixed length piston rod 40 is moved in the proximal direction 22.

Thereby, the fixed length piston rod 40 is moved by a predefined distance relative to the bung 5. This distance is defined by the compression length of the spring member 38.

The spring member 38 thereby ensures that the piston rod 40 is not in permanent abutment with the bung 5. Instead, after the adjustment of the length of the piston rod 40 is completed, the piston rod 40 is withdrawn from the bung 5 by the predefined distance such that no force is exerted on the bung 5. Accordingly, it is prevented that fluid drips out of the cartridge when a needle is attached to the cartridge.

As the piston rod 40 now has a predefined distance to the bung 5, a priming step is no longer necessary.

However, it is not necessary for the adjustment of the length of the piston rod 40 that the drive member 11 is pushed in the distal direction 21 during assembly.

After the adjustment of the length of the piston rod 40 has been completed, the drive assembly 3 is in its second state. Now the assembly of the drive assembly 3 and the cartridge holder 41 is inserted into the body 2. Afterwards the button 51 is attached to the proximal end of the drug delivery device 1. Now the drug delivery device 1 is in its fully-assembled state.

### REFERENCE NUMERALS

1 drug delivery device
2 body
3 drive assembly
4 cartridge
5 bung
6 distance between drive assembly and cartridge
7 first piston rod member
8 second piston rod member
9 adjustment member
10 nut
11 drive member
12 longitudinal axis
13 first connection member of the adjustment member 9
14 further connection member of the first piston rod member
21 distal direction
22 proximal direction
27 second connection member of the adjustment member 9
29 further connection member of the second piston rod member
31 connection member of the first piston rod member
33 connection member of the second piston rod member
35 bearing
36 thread
37 second thread
38 spring member
39 first thread
40 piston rod
41 cartridge holder
42 cap
43 first engagement member
44 second engagement member
45 fixed member
46 moveable member
47 protrusion
48 opening
49 recess
50 projection
51 button
52 inward facing protrusion
53 outward facing protrusion

1. An assembly for a drug delivery device (1),
the assembly comprising:
a drive assembly (3) and a cartridge holder (41),
wherein the drive assembly (3) comprises a first engagement member (43) and the cartridge holder (41) comprises a second engagement member (44),
wherein the drive assembly (3) is configured to be arranged inside a body (2) of the drug delivery device (1), and
wherein the drive assembly (3) is engageable to the cartridge holder (41) by an engagement of the first and the second engagement member (43, 44).

2. The assembly of claim 1,
wherein the drive assembly (3) comprises a piston rod (40) comprising a first piston rod member (7) and a second piston rod member (8), and
wherein in a first state of the drive assembly (3), the first and the second piston rod member (7, 8) are moveable with respect to each other to adjust the length of the piston rod (40).

3. The assembly of claim 1 or 2,
wherein the drive assembly (3) comprises a fixed member (45) which is configured to be not moveable relative to the cartridge holder (41) when the first and the second engagement members (43, 44) are engaged.

4. The assembly of one of the previous claims,
wherein the drive assembly (3) comprises a moveable member (46) which is configured to be moveable relative to the cartridge holder (41) when the first and the second engagement members (43, 44) are engaged.

5. The assembly of claims 3 and 4,
wherein the moveable member (46) is threadedly connected to the fixed member (45).

6. The assembly of one of the previous claims,
wherein the drive assembly (3) comprises a member being at least partially transparent and comprising the first engagement member (43).

7. The assembly of one of the previous claims,
wherein the first and the second engagement members (43, 44) are configured to be positively locked to each other.

8. The assembly of one of the previous claims,
wherein one of the first or the second engagement member (43, 44) comprises an opening (48), and
wherein the other of the first and the second engagement member (43, 44) comprises a protrusion (47) adapted to engage into the opening (48) of the one of the first or the second engagement member (43, 44).

9. The assembly of one of the previous claims,
wherein the first and the second engagement member (43, 44) are configured to form a bayonet engagement.

10. A drug delivery device (1),
comprising an assembly according to one of the previous claims and a body (2), wherein the body (2) accommodates the drive assembly (3).

11. A method for assembling a drug delivery device (1),
wherein the method comprises the steps of:
providing a drive assembly (3) and a cartridge holder (41), wherein the drive assembly (3) is configured to be arranged inside a body (2) of the drug delivery device (1), and wherein the drive assembly (3) comprises a first engagement member (43) and the cartridge holder (41) comprises a second engagement member (44), and
engaging the drive assembly (3) to the cartridge holder (41) by an engagement of the first and the second engagement member (43, 44).

12. The method of claim 11,
wherein the drive assembly (3) comprises a piston rod (40) comprising a first piston rod member (7) and a second piston rod member (8), wherein in a first state of the drive assembly (3), the first and the second piston rod member (7, 8) are moveable with respect to each other to adjust the length of the piston rod (40), and
wherein the method further comprises the step of:
adjusting the length of the piston rod (40) after engaging the drive assembly (3) to the cartridge holder (41).

13. The method of claim 12,
wherein a cartridge (4) comprising a bung (5) is arranged inside the cartridge holder (41), and
wherein the step of adjusting the length of the piston rod (40) comprises the sub-step of fixing the first piston rod member (7) to the second piston rod member (8) when the distance (6) between the piston rod (40) and the bung (5) equals a predefined value or when the piston rod (40) abuts the bung (5), thereby forming a fixed length piston rod (40).

14. The method of claim 12 or 13,
wherein the step of adjusting the length of the piston rod (40) comprises the sub-step of visually controlling the distance (6) between the first piston rod member (7) and the bung (5).

15. The method of one of claims 12 to 14,
further comprising the steps of:
providing a body (2), and
arranging the drive assembly (3) in the body (2) after the length of the piston rod (40) has been adjusted.
